(19) United States
(12) Patent Application Publication
(10) Pub. No.: US 2026/0219224 A1
(43) Pub. Date: Jul. 30, 2026

(54) SENSOR SYSTEM

(71) Applicant: Robert Bosch GmbH, Stuttgart (DE)

(72) Inventors: Bernd Schumann, Rutesheim (DE); Christoph Schelling, Stuttgart (DE)

(21) Appl. No.: 19/143,057

(22) PCT Filed: Dec. 18, 2023
(86) PCT No.: PCT/EP2023/086343

§ 371 Date: Jun. 25, 2025

(30) Foreign Application Priority Data
Jan. 11, 2023 (DE) 10 2023 200 176.4

(51) Int. Cl. G01N 27/12; G01N 33/00

(52) U.S. Cl. CPC G01N 27/124; G01N 33/005

(57) ABSTRACT

A method for operating a sensor system, in particular a gas sensor system. The sensor system includes a sensor element having a heating element and includes a control and read-out unit for the sensor element. The control and read-out unit may include a computing unit and a memory. The method may be carried out by the control and read-out unit. Operating points to be set are executed for a measurement, in the case of which operating points the sensor element is brought to different temperatures by means of the heating element. For each operating point, a measured value is recorded. The operating points to be set are set according to a criterion.

### FIELD

The present invention relates to a method for operating a sensor system and to a sensor system.

### BACKGROUND INFORMATION

Certain sensor systems, in particular gas sensor systems, having a metal oxide as sensor material are described in the related art. However, such sensor systems have unsatisfactory long-term drift stability. This presents a challenge to reliability in use. Thus, the attainable accuracy of the sensor systems and their minimum detection threshold are limited. This may be a problem, in particular for safety critical applications. Recalibrations of the sensors during operation are complex to perform and are not always possible. A sensor for combustible gases for example, even when operated without additional exposure to gas to be detected, displays its response to the global hydrogen concentration of 0.6 ppm present in the earth's atmosphere. This hydrogen concentration thus results in a background signal of, for example, a gas sensor sensitive to hydrogen.

U.S. Patent Application Publication No. US 2020/386,728 A1 describes a method for a baseline correction. Models for drift composed of numerical elements may be used, but they do not model the properties of a sensor sufficiently accurately. Accordingly, like the gas sensor, these models age. In particular, the gas sensor is not always exposed only to clean air at switch-on, which means that a baseline correction cannot be reliably implemented.

### SUMMARY

An object of the present invention is to provide an improved method for operating a sensor system. Another object of the present invention is to provide an improved sensor system. These objects are achieved by certain features of the present invention. Advantageous developments and example embodiments of the present invention are disclosed herein.

A sensor system according to the present invention allows the control and reading of at least one sensor element under different measurement conditions (for example, gas exposures) and the processing of the obtained measurement signals by means of a computation method in order to ascertain a gas concentration or a gas composition of different gases. Gases negatively influencing the measurement are usually already present in the background. It is therefore not often the case that a sensor is switched on under very defined, clean ambient conditions and an accurate baseline can then first be stored. This requires a new method for forming measurement results, as described below.

According to an example embodiment of the present invention, this method provides mathematically newly calculated measurement results corrected for the background signals. Thus, the reliable determination of the gas concentration(s) is possible even under conditions in which the sensor is switched on while already in the presence of background gases of natural or anthropogenic origin. Therefore, the sensor can more reliably and dependably perform its task of, for example, detecting concentrations of combustible gases. The measurement method according to the present invention allows compensation of disturbances due to long-term drift effects in the measured values during operation, wherein sensor signals are obtained under different measurement conditions and are processed with each other so that a gas concentration or a gas composition can be ascertained as compensated gas concentration(s).

According to a first aspect, the present invention relates to a method for operating a sensor system, in particular a gas sensor system. According to an example embodiment of the present invention, the sensor system includes a sensor element having a heating element and comprises a control and read-out unit for the sensor element. The control and read-out unit may comprise a computing unit and a memory. The method may be carried out by the control and read-out unit. Operating points to be set are executed for a measurement, in the case of which operating points the sensor element is brought to different temperatures by means of the heating element. For each operating point, a measured value is recorded. The operating points to be set are set according to a criterion.

This constitutes, for example, a self-learning adaptive sensor system which operates one or more sensors, in particular gas sensors. This sensor system with connected gas sensors is capable of detecting, by means of a particular operating mode of the sensor (this operating mode being provided, for example, by a computing unit of the control and read-out unit), under what conditions the gas sensor is operated at switch-on and whether it is switched on in a range of higher or lower measurable gas concentrations. The sensor system may have a memory in which measured values or also measurement results from previous measurements can be stored in the form of tensors. A tensor may be configured according to the definition that is common in mathematics. In particular, via a matrix of measured values, a tensor may represent the matrix elements or a subset thereof as a sum, i.e., a scalar or a vector, the components of which are partial sums. In addition, the sensor system has a computing unit by means of which present measured values can be processed with past ones.

In particular, according to an example embodiment of the present invention, the sensor system, in order to operate the sensor to be measured, sets two or more operating temperatures of the sensor via the heating element. It may be provided that, at each temperature, a direct voltage and/or an alternating voltage and/or a variable voltage is applied to the sensor.

The sensor elements used in such sensor systems may have, for example, a quasi-logarithmic characteristic curve in which a sensor element resistance is in a logarithmic relationship with the operating temperature. For certain operating temperatures, it may be assumed that the gas to be determined is no longer adsorbed on the sensor element. Thus, at high operating temperatures, a deviation of the sensor element resistance may be attributed to a change in the sensor element. This allows a deviation of the sensor element resistance at low operating temperatures to be attributed either to a change in the sensor element, if deviations are also present at high operating temperatures, or to an adsorbed gas, if no deviations are present at high operating temperatures. The optional embodiments discussed below allow this differentiation. If the sensor element changes, for example due to damage, aging or other influences, the characteristic curve also changes.

One criterion may be a different gas sensitivity of a gas sensor element, or of multiple gas sensor elements which are located in a sensor system, to different gases in the case of different operating temperatures. In particular, lower operating temperatures at which the gas sensitivity of the gas sensor elements is not yet so pronounced or higher operating temperatures at which a gas sensor element indicates the gases less strongly, barely still indicates them, or no longer indicates them at all and thus has a sensitivity reduced by the higher operating temperature may also be set.

According to an example embodiment of the present invention, another criterion may be a different gas sensitivity of a gas sensor element, or of multiple gas sensor elements which are located in a sensor system, to different gases in the case different temperatures with the application of different voltages or currents. The voltages may in particular be direct voltages or alternating voltages, for example square-wave voltages or sine-wave voltages or other alternating voltages, to which an offset voltage may also be added.

According to a second aspect, the present invention relates to a sensor system, in particular a gas sensor system. The sensor system comprises a sensor element having a heating element and comprises a control and read-out unit for the sensor element.

The control and read-out unit is configured to carry out the method according to the present invention and to control the sensor element accordingly.

Example embodiments of the method according to the present invention are explained below. In particular, these can each likewise be carried out by means of the control and read-out unit.

In one example embodiment of the method of the present invention, the operating points to be set also comprise a voltage applied to the sensor element. This voltage may be a direct voltage and/or an alternating voltage and may also comprise a polarity reversal of the direct voltage and/or alternating voltage, as well as a combination thereof.

When a direct voltage is applied, a sensor element resistance is determined by measuring the current through the sensor element. The polarity of the sensor element may also be reversed when the direct voltage is applied, for example by means of electronic switches. Thus, the current can be measured in two directions through the sensor element, if, for example, a sensor material also contains charge carriers of very low mobility (e.g., ions) that influence the conductivity when a direct voltage is applied over a certain time.

According to an example embodiment of the present invention, when an alternating voltage is applied, in particular a sinusoidal alternating voltage at different frequencies, a complex impedance of the sensor element is determined by measuring the current and the phase between current and voltage. Likewise, in the case of an alternating voltage, a direct voltage offset may also be additionally applied, and the polarity of the direct voltage offset may also be reversed by means of switches or electronic devices.

It is also possible to apply a sum of multiple sinusoidal or other variable voltages such as square-wave voltages, or other forms of voltage pulses. Thus, currents that can be attributed to impedances and resistances can likewise be measured.

The application of the different voltages may be associated with a time program for setting the different temperatures. In this case, for each set temperature, a coordinated program for setting the voltages and obtaining the measured values is carried out.

Thus, according to an example embodiment of the present invention, the method may in particular include combining program steps for setting temperatures and program steps for setting voltages, and obtaining the resulting measured values.

In this case, it may be provided that a set of operating points i is executed which consists of a combination of particular voltage and temperature program steps APi. For each operating point i, a set of measured values APi (Mi) is then obtained. The index i increases continually with increasing number of completed operating points.

In one example embodiment of the method of the present invention, the criterion includes selecting time points on the basis of an elapsed time period and choosing time points of the operating points to be set according to the elapsed time period. The time points of the operating points i of the system to be set may then be selected, for example, according to a method in which time points are determined according to an elapsed time period, for example a particular time interval, such as a day, a week, or for example also three hours or other time intervals.

In one example embodiment of the method of the present invention, the criterion includes selecting time points on the basis of a defined function and choosing time points of the operating points to be set according to the defined function.

The defined function may be, for example, continuously increasing and may comprise, for example, a logarithmically increasing interval. The time points may, for example, lie at hours such as 1 h, 5 h, 10 h, 50 h, 100 h, and so on.

In one example embodiment of the method of the present invention, the control and read-out unit comprises the aforementioned memory. The memory stores previous measured values for operating points of a previous operating state. Present measured values of a present operating state are compared to the previous measured values. A characteristic measure may be introduced for this purpose. The characteristic measure may be a scalar or a vector with components or also a matrix. The measure may comprise an assignment of numerical values formed by functions to one or more measured values for selected operating states. The measured values are arguments of these functions. The characteristic measure may be, for example, the difference between a present measured value and a stored earlier measured value or measured value determined during manufacture of the sensor system. It may be, for example, the sum of the differences of multiple measured values or a weighted sum of the differences of multiple measured values. A weighted sum does not simply add up every summand but rather adds up the summands multiplied by a number not equal to 1. The characteristic thus describes deviations of measured values from a previous measured value by numbers. In the comparison of present and previous measured values, it is checked whether a characteristic measure of the present measured values is within a first tolerance range or outside the first tolerance range and within a second tolerance range or outside the second tolerance range.

In particular, according to an example embodiment of the present invention, it may be checked whether the characteristic measure is outside the first tolerance range and within the second tolerance range, since for this case a virtual operating point with virtual measured values is ascertained and the characteristic measure is adjusted on the basis of the virtual operating point. In a new measurement, the virtual operating point may be used alternatively or additionally.

The characteristic measure can describe the aforementioned change in the characteristic curve due to a change in the sensor element.

This makes it possible, for example, to select an operating point on the basis of an event resulting from an evaluation of the measured values Mᵢ of a particular operating point APᵢ. The event results, for example, from the detection of a particular deviation from other operating points to be compared, for example the AP_{jF} (factory measured values). During manufacture of the sensor system, it is possible, for example, to set certain operating points that determine the sensitivity of the gas sensor element to certain gases to be detected, in dependence on the operating temperature (e.g., 250° C., 300° C., 500° C.). These may then be used later for comparison with real measured values at the same operating temperatures. In particular, an increase or decrease in the gas sensitivity of the sensor element for the APF measured values at different operating temperatures may be used to perform a correction of the sensor signals.

Thus, the time points of the determination of the measured values Mᵢ at an operating point APᵢ may be regular or triggered by a function or by an event. An event may be, for example, a sensor resistance that suddenly increases or decreases sharply. In one example embodiment of the method of the present invention, a measurement result is output in the event that the characteristic measure of the present measured values is within the first tolerance range.

In one example embodiment of the method of the present invention, in the event that the characteristic measure is outside the first tolerance range and within the second tolerance range, a new operating state is introduced, which is referred to as a functional measurement test. In this operating state (which may be referred to as a functional test measurement operating state), it can be assumed that the gas sensor element could function but now has a somewhat altered characteristic curve and/or a somewhat altered gas sensitivity and/or a somewhat altered base resistance. A series of operating temperatures of the gas sensor element may now be set, the associated measured values may be obtained, and the characteristic measure may be obtained. A new virtual operating point is defined therefrom, and for the future operation of the sensor system the characteristic measure of the future present measured values is calculated on the basis of the new virtual operating point of the functional measurement test. On the basis of this functional measurement test, which can also be carried out several times, a new first tolerance range and a new second tolerance range can now be defined. The old values for the first tolerance range and the second tolerance range may additionally remain stored for comparison.

In one example embodiment of the method of the present invention, an error message is output in the event that the characteristic measure of the present measured values is outside the second tolerance range.

In one example embodiment of the method of the present invention, the previous operating state includes a functional test measurement operating state during a functional test measurement. The first tolerance range and the second tolerance range may be newly defined.

In one example embodiment of the method of the present invention, in particular the following steps are carried out:
reading present measured values of the sensor element ascertained at an operating point of the sensor element;
calculating the characteristic measure of the present measured values on the basis of the operating point;
checking whether the characteristic measure is in the first specified tolerance range;
outputting a measurement result ascertained from the present measured values, if the characteristic measure is in the first specified tolerance range;
checking whether the characteristic measure is in the second specified tolerance range, if the characteristic measure is not in the first specified tolerance range;
outputting an error message, if the characteristic measure is not in the second specified tolerance range;
ascertaining a new virtual operating point and calculating a characteristic measure of the present measured values on the basis of the new virtual operating point;
newly determining a new first specified tolerance range and a new second specified tolerance range on the basis of the new virtual operating point;
checking again whether the characteristic measure of the present measured values is in the new first specified tolerance range and/or in the new second specified tolerance range;
if necessary, repeating the last two steps a second time.

The repetition may be performed several times, optionally until a specified termination criterion. In particular, a maximum number of repetitions may be specified, for example a maximum of five repetitions.

In particular, present measured values APᵢ₀ (Mᵢ₀) denoted with it thus may be compared with the measured values AP_{jF} (M_{jF}) which were ascertained in a functional test measurement during manufacture of the sensor system. Continuously during operation of the sensor system, at a later selected time point tx further operating points APᵢ₊ₜₓ (Mᵢ₊ₜₓ) of a present operating state APᵢ₀ (Mᵢ₀) are compared again to the measured values AP_{jF} (M_{jF}) and also additionally to operating states that the sensor has previously completed, i. e., to operating points APₖ₋ₜₓ (Mₖ₋ₜₓ) which (indicated by −tx) preceded the present APᵢ₀ (Mᵢ₀) operating state. Thus, the sensor system may compare the measured values of present operating states to historical measured values prior to the present operating state as well as to measured values which were ascertained in the functional test measurement.

The measured values of the operating points APᵢ₀ and the differences of the measured values of these operating points APᵢ₀ (Mᵢ₀) from the measured values AP_{jF} (M_{iF}) and from the previous measured values in APₖ₋ₜₓ (Mₖ₋ₜₓ) are then stored in a continuous series of order structures such as vectors or matrices (generally: tensors). These different order structures thus consist of the stored measured values Ord_{iM} and Ord_{iM,Diff}, Which include the difference matrices or vectors. The characteristic measure may also be represented therein or calculated from these data according to a rule.

The order elements of an operating point now consist of the measured values and the differences of the measured values of selected individual operating points, and a structure Ord_{iM,Diff} always contains the present measured values and the differences of at least two operating points. On the structure Ord_{iM,Diff}, characteristic measures MAᵢ are now defined, which may be formed multiple times.

According to an example embodiment of the present invention, the characteristic measures MAi may map the differences of all measured values in the structure Ord_{iM}, Diff to a sum MA_{Sum_i0,k}.

Here, i0 is the index of the operating point of the present operating state APᵢ₀ and k is the index of another selected operating state APₖ. Alternatively, the characteristic measures MAᵢ may map the differences of some selected measured values of an operating point APᵢ, for example only in a certain range of the measured values (this may be, for example, a certain resistance range or impedance range), to a sum. In this way, it can be ascertained whether certain measured values occur often or rather rarely in multiple operating points.

Characteristic measures of the type MAᵢ are always associated with exactly one operating point APᵢ. In principle, additional characteristic measures MA_{T,U} which map the differences of a subset of the measured values, for example only at certain temperatures or voltages, may also be introduced. These characteristic measures are always associated with certain voltages Uₙ and/or temperatures Tₘ and thus extend across multiple operating points APᵢ. . . APₖ. The characteristic measures may be, for example, sums, weighted sums or functions on the measured values and or on the differences of the measured values with respect to earlier operating points.

A catalog which the sensor system can always access is set up for all measured values of the APᵢ, the Ord_{iM} and the differences Ord_{iM,Diff} and the characteristic measures MAᵢ and MA_{T,U} . It is also possible to store only a portion of this information, for example only the characteristic measures, in the catalog in order to save memory space.

On the basis of the order structures and characteristic measures stored in the catalog, a curve VerMA of the characteristic measures MAᵢ and MA_{T,U} may be created. The curve of the characteristic measures indicates the rise or fall or a constant course of the value of the particular selected characteristic measures of the different operating points APᵢ. If a particular characteristic measure exceeds a specified first tolerance range TB₁, which may be formed with the measured values and order structures of the AP_{jF} (M_{iF}), it is checked whether this characteristic measure is now within a second tolerance range TB₂. If it is within this second tolerance range TB₂, the new virtual operating point APᵥᵢᵣₜ₁ with virtual measured values is formed by means of an adaptation function, and this new virtual operating point is then always additionally included, as a new reference operating point AP_{ref}, in the forming of the characteristic measure. The respective operating points APᵢ and their order structures then form their characteristic measures and the order structures such that the past operating points as well as the additional operating point AP_{ref} are likewise represented in the order structures.

This step makes possible an adaptation method carried out using the tolerance ranges TB₁ and TB₂. If a sensor element is always within the second tolerance range TB₂, its measured values can be corrected with the virtual measured values of the virtual operating point; the correction may be simply linear or logarithmic or according to another function. After the correction, the sensor element can then be back within the first tolerance range TB₁ which is then newly created.

If a sensor element with its characteristic measures is outside the second tolerance range TB₂, its measured values are classified as erroneous. Further tolerance bands may also be used, in addition to the tolerance bands TB₁ and TB₁, for measured values and characteristic measures, for example for a subset of, for example, selected measured values of a particular range or within a particular temperature or voltage.

Embodiment examples of the present invention are explained with reference to the figures.

### BRIEF DESCRIPTION OF THE DRAWINGS

FIG. 1 shows a sensor system according to an example embodiment of the present invention.

FIG. 2 shows a flowchart of a method for operating a sensor system, according to an example embodiment of the present invention.

### DETAILED DESCRIPTION OF EXAMPLE EMBODIMENTS

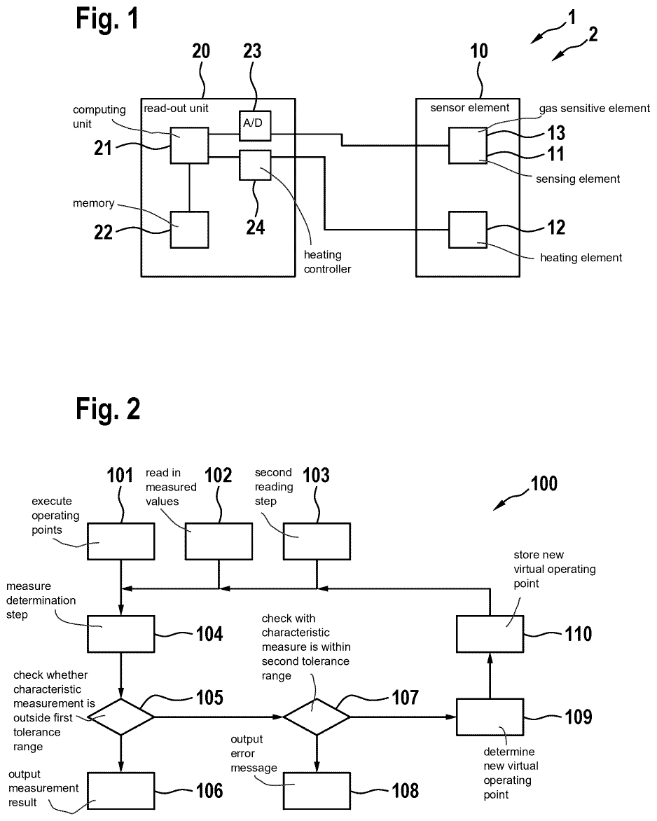

FIG. 1 shows a sensor system 1, in particular a gas sensor system 2. The sensor system 1 comprises a sensor element 10. The sensor element 10 comprises a sensing element 11, by means of which a physical quantity can be converted into an electronic signal. The sensor element 10 further comprises a heating element 12. If the sensor system 1 is the gas sensor system 2, the sensing element 11 may be configured as a gas-sensitive element 13. The sensor system 1 further comprises a control and read-out unit 20 for the sensor element 10. The control and read-out unit 20 may in particular comprise a computing unit 21 and a memory 22. The control and read-out unit 20 may also comprise, for example, an analog-to-digital converter 23 by means of which a signal from the sensing element 11 can be converted into a digital signal. In addition, the control and read-out unit 20 may comprise, for example, a heating controller 24, by means of which the heating element 12 can be controlled and in particular a temperature can be specified for the sensor element 10 or the sensing element 11. It may be provided that the control and read-out unit 20 is configured to carry out the method described below and to control the sensor element 10 accordingly.

The sensor elements 10 used in such sensor systems 1 may have, for example, a quasi-logarithmic characteristic curve in which a sensor element resistance is in a logarithmic relationship with the operating temperature. For certain operating temperatures, it may be assumed that the gas to be determined is no longer adsorbed on the sensor element 10. Thus, at high operating temperatures, a deviation of the sensor element resistance may be attributed to a change in the sensor element 10. This allows a deviation of the sensor element resistance at low operating temperatures to be attributed either to a change in the sensor element 10, if deviations are also present at high operating temperatures, or to an adsorbed gas, if no deviations are present at high operating temperatures.

FIG. 2 shows a flowchart 100 of a method of operating a sensor system, for example the sensor system 1 of FIG. 1. In particular, the control and read-out unit 20 of the sensor system 1 of FIG. 1 may be configured to carry out this method.

In an execution step 101, operating points to be set are executed for a measurement, in the case of which operating points the sensor element 10 or the sensing element 11 is brought to different temperatures by means of the heating element 12. For each operating point, a measured value is recorded. The operating points to be set are set according to a criterion.

One criterion may be a different gas sensitivity of a gas sensor element 10, or of multiple gas sensor elements 10 which are located in a sensor system 1, to different gases in the case of different operating temperatures. In particular, lower operating temperatures at which the gas sensitivity of the gas sensor elements 10 is not yet so pronounced or higher operating temperatures at which a gas sensor element 10 indicates the gases less strongly, barely still indicates them, or no longer indicates them at all and thus has a sensitivity reduced by the higher operating temperature may also be set.

Another criterion may be a different gas sensitivity of a gas sensor element 10, or of multiple gas sensor elements 10 which are located in a sensor system 1, to different gases in the case different temperatures with the application of different voltages or currents. The voltages may in particular be direct voltages or alternating voltages, for example square-wave voltages or sine-wave voltages or other alternating voltages, to which an offset voltage may also be added.

In one embodiment of the method, the operating points to be set also comprise a voltage applied to the sensor element 10 or to the sensing element 11. This voltage may be a direct voltage and/or an alternating voltage and may also comprise a polarity reversal of the direct voltage and/or alternating voltage, as well as a combination thereof.

When a direct voltage is applied, a sensor element resistance is determined by measuring the current through the sensor element 10, or a sensing element resistance is determined by measuring the current through the sensing element 11. The polarity of the sensor element 10 or the sensing element 11 may also be reversed when the direct voltage is applied, for example by means of electronic switches. Thus, the current can be measured in two directions through the sensor element 10 or sensing element 11, if, for example, a sensor material also contains charge carriers of very low mobility (e.g., ions) that influence the conductivity when a direct voltage is applied over a certain time.

When an alternating voltage is applied, in particular a sinusoidal alternating voltage at different frequencies, a complex impedance of the sensor element 10 or the sensing element 11 is determined by measuring the current and the phase between current and voltage. Likewise, in the case of an alternating voltage, a direct voltage offset may also be additionally applied, and the polarity of the direct voltage offset may also be reversed by means of switches or electronic devices. The switches and/or electronic devices may be part of the control and read-out unit 20.

It is also possible to apply a sum of multiple sinusoidal or other variable voltages such as square-wave voltages, or other forms of voltage pulses. Thus, currents that can be attributed to impedances and resistances can likewise be measured.

The application of the different voltages may be associated with a time program for setting the different temperatures. In this case, for each set temperature, which is then approached by means of the heating element 12, a coordinated program for setting the voltages and obtaining the measured values is carried out.

Thus, the method may in particular include combining program steps for setting temperatures and program steps for setting voltages, and obtaining the resulting measured values. In this case, it may be provided that a set of operating points i is executed which consists of a combination of particular voltage and temperature program steps APᵢ. For each operating point i, a set of measured values APᵢ (Mᵢ) is then obtained. The index i increases continually with increasing number of completed operating points.

In one embodiment example of the method, the criterion includes selecting time points on the basis of an elapsed time period and choosing time points of the operating points to be set according to the elapsed time period. The time points of the operating points i of the system to be set may then be selected, for example, according to a method in which time points are determined according to an elapsed time period, for example a particular time interval, such as a day, a week, or for example also three hours or other time intervals.

In one embodiment example of the method, the criterion includes selecting time points on the basis of a defined function and choosing time points of the operating points to be set according to the defined function. The defined function may be, for example, continuously increasing and may comprise, for example, a logarithmically increasing interval. The time points may, for example, lie at hours such as 1 h, 5 h, 10 h, 50 h, 100 h, and so on.

In one embodiment example of the method, the control and read-out unit 20 comprises the memory 22. The memory 22 stores previous measured values for operating points of a previous operating state. Present measured values of a present operating state are compared to the previous measured values. A characteristic measure may be introduced for this purpose. The characteristic measure may be a scalar or a vector with components or also a matrix. The measure may comprise an assignment of numerical values formed by functions to one or more measured values for selected operating states. The measured values are arguments of these functions. The characteristic measure may be, for example, the difference between a present measured value and a stored earlier measured value or measured value determined during manufacture of the sensor system. It may be, for example, the sum the differences of multiple measured values or a weighted sum of the differences of multiple measured values. A weighted sum does not simply add up every summand but rather adds up the summands multiplied by a number not equal to 1. The characteristic thus describes deviations of measured values from a previous measured value by numbers. It is checked whether the characteristic measure of the present measured values is within a first tolerance range or outside the first tolerance range and within a second tolerance range or outside the second tolerance range.

In particular, it may be checked whether the characteristic measure is outside the first tolerance range and within the second tolerance range, since for this case a virtual operating point with virtual measured values is ascertained and the characteristic measure is adjusted on the basis of the virtual operating point. In a new measurement, the virtual operating point may be used alternatively or additionally.

The characteristic measure may be determined, for example, in a measure determination step 104. Checking whether the characteristic measure is outside the first tolerance range may be done in a first decision step 105. Checking whether the characteristic measure is within the second tolerance range may be done in a second decision step 107. Ascertaining a new virtual operating point may be done in a determining step 109.

With the new virtual operating point, a new first specified tolerance range and a new second specified tolerance range can be determined and the first decision step 105 can be performed with the new first specified tolerance range and the second decision step 107 can be performed with the new second specified tolerance range.

This makes it possible, for example, to select an operating point on the basis of an event resulting from an evaluation of the measured values Mᵢ of a particular operating point APᵢ. The event results, for example, from the detection of a particular deviation from other operating points to be compared, for example AP_{jF} (factory measured values). The measured values determined for the operating point APJF to be compared may be read in a first reading step 102. During manufacture of the sensor system 1, it is possible, for example, to set certain operating points that determine the sensitivity of the gas sensor element 10 to certain gases to be detected, in dependence on the operating temperature (e. g., 250° C., 300° C., 500° C.). These may then be used later for comparison with real measured values at the same operating temperatures. In particular, an increase or decrease in the gas sensitivity of the sensor element 10 for the AP_{jF} measured values at different operating temperatures may be used to perform a correction of the sensor signals.

Thus, the time points of the determination of the measured values Mᵢ at an operating point APᵢ may be regular or triggered by a function or by an event.

In one embodiment example of the method, a measurement result is output in the event that the characteristic measure of the present measured values is within the first tolerance range.

This may be done, for example, in a measurement result output 106.

In one embodiment example of the method, an error message is output in the event that the characteristic measure of the present measured values is outside the second tolerance range. This may be done, for example, in an error message output 108.

In one embodiment example of the method, the previous operating state includes a functional test measurement operating state during a functional test measurement.

In one embodiment example of the method, in the event that the characteristic measure is outside the first tolerance range and within the second tolerance range, a new operating state is introduced, which may be referred to as a functional measurement test. In this operating state (which may be referred to as a functional test measurement operating state), it can be assumed that the gas sensor element could function but now has a somewhat altered characteristic curve and/or a somewhat altered gas sensitivity and/or a somewhat altered base resistance. A series of operating temperatures of the gas sensor element 10 may now be set, the associated measured values may be obtained, and the characteristic measure may be obtained. A new virtual operating point is defined therefrom, and for the future operation of the sensor system the characteristic measure of the future present measured values is calculated on the basis of the new virtual operating point of the functional measurement test.

On the basis of this functional measurement test, which can also be carried out several times, a new first tolerance range and a new second tolerance range can now be defined. The old values for the first tolerance range and the second tolerance range may additionally remain stored for comparison.

In particular, present measured values APᵢ₀ (Mᵢ₀) denoted with i0, ascertained in the execution step 101, thus may be compared with measured values AP_{jF} (M_{iF}) which were ascertained in a functional test measurement during manufacture of the sensor system and which are read in the first reading step 102. Continuously during operation of the sensor system, at a later selected time point tx further operating points APᵢ₊ₜₓ (Mᵢ₊ₜₓ) of a present operating state APᵢ₀ (Mᵢ₀) are compared again to the measured values AP_{jF} (M_{jF}) denoted with jF and also additionally to operating states that the sensor has previously completed, i. e., to operating points APₖ₋ₜₓ (Mₖ₋ₜₓ) which (indicated by −tx) preceded the present APᵢ₀ (Mᵢ₀) operating state. These may be read in a second reading step 103. Thus, the sensor system 1 may compare the measured values of present operating states to historical measured values prior to the present operating state as well as to measured values which were ascertained in the functional test measurement.

The measured values of the operating points APᵢ₀ and the differences of the measured values of these operating points APᵢ₀ (Mᵢ₀) from the measured values AP_{jF} (M_{iF}) and from the previous measured values in APₖ₋ₜₓ (Mₖ₋ₜₓ) are then stored in a continuous series of order structures such as vectors or matrices (generally: tensors). These different order structures thus consist of the stored measured values Ord_{iM} and Ord_{iM,Diff}, which include the difference matrices or vectors.

The order elements of an operating point now consist of the measured values and the differences of the measured values of selected individual operating points, and a structure Ord_{iM,Diff} always contains the present measured values and the differences of at least two operating points. On the structure Ord_{iM,Diff}, characteristic measures MAᵢ are now defined, which may be formed multiple times.

The characteristic measures MAᵢ may map the differences of all measured values in the structure Ord_{iM,Diff} to a sum MA_{Sum i0,k}. £ Here, i0 is the index of the operating point of the present operating state APᵢ₀ and k is the index of another selected operating state APₖ. Alternatively, the characteristic measures MAᵢ may map the differences of some selected measured values of an operating point APᵢ, for example only in a certain range of the measured values (this may be, for example, a certain resistance range or impedance range), to a sum. In this way, it can be ascertained whether certain measured values occur often or rather rarely in multiple operating points.

Characteristic measures of the type MAᵢ are always associated with exactly one operating point APᵢ. In principle, additional characteristic measures MA_{T,U} which map the differences of a subset of the measured values, for example only at certain temperatures or voltages, may also be introduced. These characteristic measures are always associated with certain voltages Un and/or temperatures Tₘ and thus extend across multiple operating points APᵢ. . . APₖ.

A catalog which the sensor system can always access is set up for all measured values of the APᵢ, the Ord_{iM} and the differences Ord_{iM,Diff} and the characteristic measures MAᵢ and MA_{T,U}. It is also possible to store only a portion of this information, for example only the characteristic measures, in the catalog in order to save memory space.

On the basis of the order structures and characteristic measures stored in the catalog, a curve VerMA of the characteristic measures MAᵢ and MA_{T,U} may be created. The curve of the characteristic measures indicates the rise or fall or a constant course of the value of the particular selected characteristic measures of the different operating points APᵢ. If a particular characteristic measure exceeds a specified first tolerance range TB₁ (ascertained in the first decision step 105), which may be formed with the measured values and order structures of the AP_{jF} (M_{iF}), it is checked whether this characteristic measure is now within a second tolerance range TB₂ (ascertained in the second decision step 107). If it is within this second tolerance range TB₂, the new virtual operating point APᵥᵢᵣₜ₁ with virtual measured values is formed by means of an adaptation function in the determining step 109, and this new virtual operating point is then always additionally included, as a new reference operating point AP_{ref}, in the forming of the characteristic measure. The new virtual operating point APᵥᵢᵣₜ₁ may be stored in the memory 22 in a storage step 110 and is available for a new execution of the measure determination step 104. The respective operating points APᵢ and their order structures then form their characteristic measures and the order structures such that the past operating points as well as the additional operating point AP_{ref} are likewise represented in the order structures.

This step makes possible an adaptation method carried out using the tolerance ranges TB₁ and TB₂. If a sensor element 10 or sensing element 11 is always within the second tolerance range TB₂, its measured values can be corrected with the virtual measured values of the virtual operating point; the correction may be simply linear or logarithmic or according to another function. After the correction, the sensor element 10 or sensing element 11 can then be back within the first tolerance range TB₁.

If a sensor element 10 or sensing element 11 with its characteristic measures is outside the second tolerance range TB₂, its measured values are classified as erroneous. Further tolerance bands may also be used, in addition to the tolerance bands TB₁ and TB₁, for measured values and characteristic measures, for example for a subset of, for example, selected measured values of a particular range or within a particular temperature or voltage.

Although the present invention has been described in detail by means of the preferred embodiment examples, the present invention is not limited to the disclosed examples and other variations may be derived therefrom by a person skilled in the art without departing from the scope of protection of the present invention.

1-10. (canceled)

11. A method for operating a sensor system, wherein the sensor system includes a sensor element having a heating element, and a control and read-out unit for the sensor element, the method comprising:
executing operating points to be set for a measurement, the sensor element being brought to different temperatures using the heating element for the operating points;
for each operating point of the operating points, recording a measured value;
wherein the operating points to be set are set according to a criterion.

12. The method according to claim 11, wherein the sensor system is a gas sensor system.

13. The method according to claim 11, wherein the operating points to be set also include a voltage applied to the sensor element.

14. The method according to claim 11, wherein the criterion includes selecting time points based on an elapsed time period and choosing time points of the operating points to be set according to the elapsed time period.

15. The method according to claim 11, wherein the criterion includes selecting time points based on a defined function and choosing time points of the operating points to be set according to the defined function.

16. The method according to claim 11, wherein the control and read-out unit includes a memory, wherein the memory stores previous measured values for operating points of a previous operating state, wherein present measured values of a present operating state are compared with the previous measured values and it is checked whether a characteristic measure of the present measured values is outside a first tolerance range and within a second tolerance range, wherein, based on the characteristic measure of the present measured values being outside the first tolerance range and within the second tolerance range, a virtual operating point with virtual measured values is ascertained and the characteristic measure is adjusted based on the virtual operating point, wherein, in a new measurement, the virtual operating point is also used.

17. The method according to claim 16, wherein a measurement result is output in the event that the characteristic measure of the present measured values is within the first tolerance range.

18. The method according to claim 16, wherein an error message is output in the event that the characteristic measure of the present measured values is outside the second tolerance range.

19. The method according to claim 16, wherein the previous operating state includes a functional test measurement operating state during a functional test measurement.

20. The method according to claim 16, further comprising the following steps:
reading present measured values of the sensor element ascertained at an operating point of the sensor element;
calculating the characteristic measure of the present measured values based on the operating point;
checking whether the characteristic measure is in the first specified tolerance range;
outputting a measurement result ascertained from the present measured values, when the characteristic measure is in the first specified tolerance range;
checking whether the characteristic measure is in the second specified tolerance range, when the characteristic measure is not in the first specified tolerance range;
outputting an error message, when the characteristic measure is not in the second specified tolerance range;
ascertaining a new virtual operating point and calculating a characteristic measure of the present measured values based on the new virtual operating point;
newly determining a new first specified tolerance range and a new second specified tolerance range based on the new virtual operating point;
checking again whether the characteristic measure of the present measured values is in the new first specified tolerance range and/or in the new second specified tolerance range; and
when necessary, repeating the newly determining and the check again steps a second time.

21. A sensor system, comprising:
a sensor element having a heating element; and
a control and read-out unit for the sensor element (10), wherein the control and read-out unit is configured to carry out a method and to control the sensor element accordingly, the method including:
executing operating points to be set for a measurement, the sensor element being brought to different temperatures using the heating element for the operating points, and
for each operating point of the operating points, recording a measured value,
wherein the operating points to be set are set according to a criterion.

22. The sensor system according to claim 21, wherein the sensor system is a gas sensor system.
